(19) United States
(12) Patent Application Publication
(10) Pub. No.: US 2020/0005914 A1
(43) Pub. Date: Jan. 2, 2020

(54) METHODS AND SYSTEMS FOR TRACKING AND REPRESENTING MEDICAL INFORMATION

(71) Applicant: VCA ANTECH, INC., Los Angeles, CA (US)

(72) Inventor: David AUCOIN, Westlake Village, CA (US)

(73) Assignee: VCA ANTECH, INC., Los Angeles, CA (US)

(21) Appl. No.: 16/567,939

(22) Filed: Sep. 11, 2019

Related U.S. Application Data
(63) Continuation of application No. 15/023,360, filed on Mar. 18, 2016, now abandoned.
(60) Provisional application No. 61/881,863, filed on Sep. 24, 2013.

(51) Int. Cl. G16H 10/60

(52) U.S. Cl. CPC G16H 10/60

(57) ABSTRACT

This disclosure relates to systems and methods for educating and empowering patients and their caregivers. In one aspect, a system for providing medical information to a user includes a healthcare provider terminal, a patient record database, a patient-diagnostic processor, and an interface. The healthcare provider terminal may enable uploading of a plurality of laboratory test results, wherein each laboratory test result measures a quantity of each of a respective plurality of substances in a patient. The patient record database may include historical laboratory test results associated with the patient. The patient-diagnostic processor may generate a plurality of informational displays based on the patient's plurality of laboratory test results and the patient's historical laboratory test results, wherein each informational display provides a visualization of a change in the quantity of the respective substance over a period of time. The interface may display the plurality of displays.

### CROSS-REFERENCE TO RELATED APPLICATIONS

This application claims priority to U.S. Provisional Patent Application No. 61/881,863, filed Sep. 24, 2013, the contents of which are incorporated by reference in their entirety.

### TECHNICAL FIELD

This disclosure relates to systems and methods for educating and empowering patients and their caregivers. More particularly, this disclosure provides systems and methods that provide an accessible visualization of a patient's laboratory test results, enabling a layperson to track the test results, identify trends in the patient's physiological parameters, and deviations in those trends over time.

### BACKGROUND

Prevention and early detection of illness are critical to maintaining good health. Traditionally, a patient's laboratory test results are one of the primary tools utilized in the prevention and early detection of illness. Because these test results are complex and difficult for a layperson to digest, a doctor (or another with formal medical training) must interpret the results to detect symptoms, diagnose illnesses, and generally monitor the patient's health. Interpretation typically involves a comparison of the test results to a “reference range” of acceptable results, which is statistically determined by the results of the greater population (or a subset of that population with comparable traits to the subject patient).

Doctors typically interpret a large number of test results for a large number of patients. Also, doctors typically interpret each individual patient's results infrequently. With infrequent testing of a large number of individuals, it is difficult for doctors to identify trends in an individual patient's unique physiology, or, more importantly, deviations from those trends. Further, although statistical averages or ranges (based on the greater population) may provide a good indicator of wellness in some instances, those averages can be misleading for an individual patient's unique physiological parameters.

The individual patient (or their primary caregiver) is best suited to monitor trends in the patient's test results because (1) they have a vested interest in those results and (2) they have a much smaller number of results to monitor. Thus, the individual patient (or their primary caregiver) can identify subtle changes in the patient's unique physiological trends. However, individual patient monitoring by patients and primary caregivers has thusfar been impractical because laboratory test results are generally too complicated for patients or their primary caregivers to digest and interpret.

Thus, no single participant in a patient's healthcare team has all the necessary characteristics to monitor for subtle changes in the patient's unique physiology: doctors generally have too many patients to identify subtle changes in an individual patient's unique trends and patients (or their primary caregivers) generally lack the medical sophistication necessary to understand complex laboratory results. Thus, trends and deviations from normal physiological parameters have been overlooked in the past, undermining prevention and early detection of illness.

### SUMMARY

Disclosed herein are systems and methods that provide informational visualizations of patients' medical test history, allowing a patient or a primary caregiver to identify trends and deviations in the test results. Additional information—such as expected ranges and test descriptions—may also empower the patient or primary caregiver to track the patient's wellness and identify potentially problematic deviations in the patient's unique physiological parameters. Thus, the systems and methods described herein beneficially engage the patient or primary caregiver in actively detecting symptoms, diagnosing illnesses, and generally monitoring health by translating traditionally boring and unintelligible statistical lab results into an accessible and meaningful resource.

Additionally, the methods and systems disclosed herein may beneficially improve healthcare professionals' role by providing a quicker and more-efficient mechanism to monitor test results. That is, the healthcare professional can leverage the patient's or primary caregiver's engagement to obtain early detection and prevention of illness, thereby reducing the advancement of illnesses when first treated and reducing the resources necessary to treat the illness.

The methods and systems described herein may be particularly beneficial to primary caregivers. A substantial challenge for primary caregivers is monitoring a patient's symptoms when that patient cannot or will not communicate such symptoms. For example, parents of very young children and pet owners must monitor for outwardly displayed signs of illness, and such signs may only appear when an illness has progressed substantially. By identifying and treating illness earlier, the methods and systems disclosed herein may beneficially reduce the resources necessary to return a patient to a healthy condition.

In one aspect, a system for providing medical information to a user includes a terminal, a database, a processor, and an interface. The healthcare provider terminal may enable uploading of a plurality of laboratory test results, wherein each laboratory test result measures a quantity of each of a respective plurality of substances in a patient. The patient record database may include historical laboratory test results associated with the patient. The patient-diagnostic processor may generate a plurality of informational displays based on the patient's plurality of laboratory test results and the patient's historical laboratory test results, wherein each informational display provides a visualization of a change in the quantity of the respective substance over a period of time. The interface may display the plurality of displays. In some aspects, the terminal may be a healthcare provider terminal, the database may be a patient-record database, and the processor may be a patient-diagnostic processor.

In a further aspect, the visualization of the change in the quantity of the respective substance may include a plurality of one-dimensional graphs of the quantity, wherein each of the plurality of one-dimensional graphs may represent a temporally distinct laboratory test result of the respective substance.

In a further aspect, each of the plurality of informational displays may include an expected range of the respective substance. In yet a further aspect, the expected range may be a function of a genetic trait of the patient.

In a further aspect, the plurality of laboratory tests may include a blood test of the patient and the plurality of substances may include at least one of total protein, albumin, globulin, A/G ratio, ALT, Alk phosphate, BUN, creatinine, BUN/creatinine ratio, glucose, and potassium. In a further aspect, the plurality of laboratory tests may include a blood test of the patient and the plurality of substances includes at least one of white blood cells, red blood cells, and platelets. In a further aspect, the laboratory tests may include urine analysis of the patient, and the plurality of indicators may include at least one of color, appearance, concentration, pH, chemical components, and the presence of microscopic particles. In a further aspect, the laboratory tests may include a heartworm analysis of the patient.

In a further aspect, an background information display may provide background information about one of the plurality of test results, wherein the background information display may be activated through the interface.

In another aspect, a computer-implemented method of providing medical information to a patient includes receiving a plurality of test results which measure a quantity of each of a respective plurality of substances in the patient, accessing historical laboratory test results associated with the patient, generating a plurality of informational displays based on the patient's plurality of laboratory test results and the patient's historical laboratory test results, and displaying the plurality of informational displays on an interface. Each informational display may provide a visualization of a change in the quantity of the respective substance over a period of time.

In a further aspect, the visualization of the change in the quantity of the respective substance may include a plurality of one-dimensional graphs of the quantity, wherein each of the plurality of one-dimensional graphs may represent a temporally distinct laboratory test result of the respective substance.

In a further aspect, the computer implemented method may include displaying an expected range of the respective substance on the interface. In yet a further aspect, the expected range may be a function of a genetic trait of the patient.

In a further aspect, the plurality of laboratory tests may include a blood test of the patient and the plurality of substances may include at least one of total protein, albumin, globulin, A/G ratio, ALT, Alk phosphate, BUN, creatinine, BUN/creatinine ratio, glucose, and potassium. In a further aspect, the plurality of laboratory tests may include a blood test of the patient and the plurality of substances may include at least one of white blood cells, red blood cells, and platelets. In a further aspect, the laboratory tests may include urine analysis of the patient, and the plurality of indicators may include at least one of color, appearance, concentration, pH, chemical components, and the presence of microscopic particles. In a further aspect, the laboratory tests may include a heartworm analysis of the patient.

In a further aspect, the computer implemented method may include displaying background information about one of the plurality of test results when the user interacts with the interface.

In yet another aspect, a non-transitory computer readable storage medium stores instructions, which when executed by a device, cause the device to perform a method of providing medical information to a user, the method including receiving a plurality of test results which measure a quantity of each of a respective plurality of substances in the patient, accessing historical laboratory test results associated with the patient, generating a plurality of informational displays based on the patient's plurality of laboratory test results and the patient's historical laboratory test results, and displaying the plurality of informational displays on an interface. Each informational display provides a visualization of a change in the quantity of the respective substance over a period of time

In a further aspect, the visualization of the change in the quantity of the respective substance may include a plurality of one-dimensional graphs of the quantity, wherein each of the plurality of one-dimensional graphs may represent a temporally distinct laboratory test result of the respective substance.

In a further aspect, the method of providing medical information may include displaying an expected range of the respective substance on the interface. In yet a further aspect, the expected range may be a function of a genetic trait of the patient.

In a further aspect, the plurality of laboratory tests may include a blood test of the patient and the plurality of substances may include at least one of total protein, albumin, globulin, A/G ratio, ALT, Alk phosphate, BUN, creatinine, BUN/creatinine ratio, glucose, and potassium. In a further aspect, the plurality of laboratory tests may include a blood test of the patient and the plurality of substances may include at least one of white blood cells, red blood cells, and platelets. In a further aspect, the laboratory tests may include urine analysis of the patient, and the plurality of indicators may include at least one of color, appearance, concentration, pH, chemical components, and the presence of microscopic particles. In a further aspect, the laboratory tests may include a heartworm analysis of the patient.

In a further aspect, the method of providing medical information may include displaying background information about one of the plurality of test results when the user interacts with the interface.

In yet another aspect, an electronic device includes a processor to execute instructions and a memory coupled with the processor to store instructions, which when executed by the processor, cause the processor to perform operations to generate an application programming interface (API) that allows an API-calling component to perform the following operations: receiving a plurality of test results which measure a quantity of each of a respective plurality of substances in the patient; accessing historical laboratory test results associated with the patient; generating a plurality of informational displays based on the patient's plurality of laboratory test results and the patient's historical laboratory test results, wherein each informational display provides a visualization of a change in the quantity of the respective substance over a period of time; and displaying the plurality of informational displays on an user interface.

In a further aspect, the visualization of the change in the quantity of the respective substance may include a plurality of one-dimensional graphs of the quantity, wherein each of the plurality of one-dimensional graphs may represent a temporally distinct laboratory test result of the respective substance.

In a further aspect, the operations may include displaying an expected range of the respective substance on the interface. In yet a further aspect, the expected range may be a function of a genetic trait of the patient.

In a further aspect, the plurality of laboratory tests may include a blood test of the patient and the plurality of substances may include at least one of total protein, albumin, globulin, A/G ratio, ALT, Alk phosphate, BUN, creatinine, BUN/creatinine ratio, glucose, and potassium. In a further aspect, the plurality of laboratory tests may include a blood test of the patient and the plurality of substances includes at least one of white blood cells, red blood cells, and platelets. In a further aspect, the laboratory tests may include urine analysis of the patient, and the plurality of indicators may include at least one of color, appearance, concentration, pH, chemical components, and the presence of microscopic particles. In a further aspect, the laboratory tests may include a heartworm analysis of the patient.

In a further aspect, the operations may include displaying background information about one of the plurality of test results when the user interacts with the interface.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A illustrates an exemplary interface including an informational display of a patient's medical laboratory results, in accordance with one embodiment.

FIG. 1B illustrates a chemistry test result display similar to the test results display of FIG. 1A and with a timeline for monitoring changes in the test results, in accordance with one embodiment.

FIG. 1C illustrates the interface of FIG. 1A with a substance out of range, in accordance with one embodiment.

FIG. 1D provides an example of an interface with a roll-over function activated, in accordance with one embodiment.

FIG. 2A illustrates another exemplary interface including an informational display of a patient's medical laboratory results, in accordance with one embodiment.

FIG. 2B illustrates a chemistry test result display similar to the test results display of FIG. 2A and with a timeline for monitoring changes in the test results, in accordance with one embodiment.

FIG. 2C illustrates the interface of FIG. 2A with a substance out of range, in accordance with one embodiment.

FIG. 2D illustrates an additional exemplary interface for a complete blood count test, with information provided on red blood cells and platelets, in accordance with one embodiment.

FIG. 2E illustrates the interface of FIG. 2D with a substance out of range, in accordance with one embodiment.

FIG. 2F depicts how a doctor may traditionally view a complete blood count test for a patient, in accordance with one embodiment.

FIGS. 2G and 2H illustrate an exemplary interface for providing complete blood count test to an end-user without formal medical training, in accordance with one embodiment.

FIGS. 3A and 3B depict an exemplary interface for informational displays of test results for a urine analysis, in accordance with one embodiment.

FIG. 4 depicts an exemplary interface for informational displays of test results for ova and parasites, in accordance with one embodiment.

FIG. 5 depicts an exemplary interface for informational displays of test results for T4 (thyronxine), in accordance with one embodiment.

FIG. 6 depicts an exemplary interface for informational displays of test results for accuplex, in accordance with one embodiment.

FIG. 7 depicts an exemplary interface for informational displays of test results for FeLV and FIV, in accordance with one embodiment.

FIG. 8 depicts an exemplary interface for informational displays of test results for heartworm, in accordance with one embodiment.

FIG. 9 depicts a system for providing medical information to a user, comprising a healthcare provider terminal, a patient record database, a patient-diagnostic processor, and an interface.

It is to be understood that other embodiments may be utilized and structural changes may be made without departing from the scope of the claimed subject matter.

To engage patients and primary caregivers in actively detecting symptoms, diagnosing illnesses, and generally monitoring health, traditionally boring and unintelligible statistical lab results are translated into an accessible and meaningful resource for the layperson. The system and methods described herein present medical test information as diagrams that require no medical training to identify trends in physiological parameters and deviations from those trends. In addition, changes in medical test results over time are depicted to offer the lay individual an opportunity to appreciate a deviation from normal trends in a patient's physiological parameters.

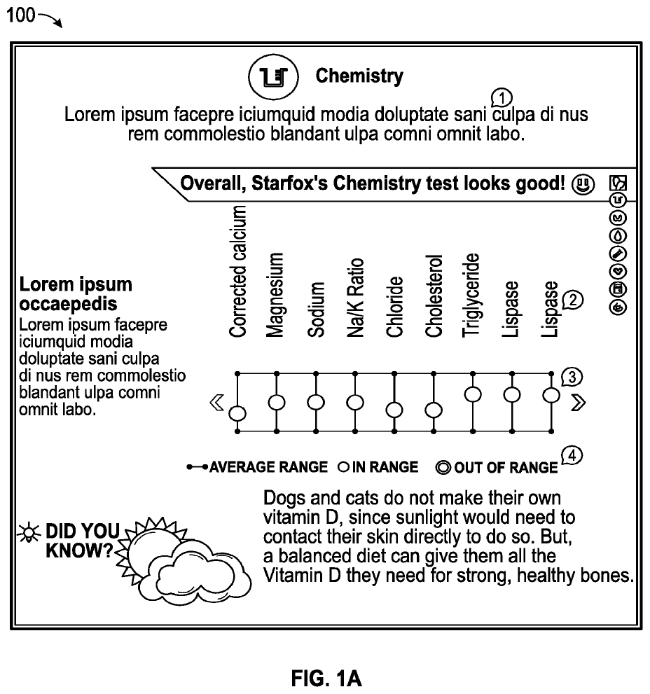

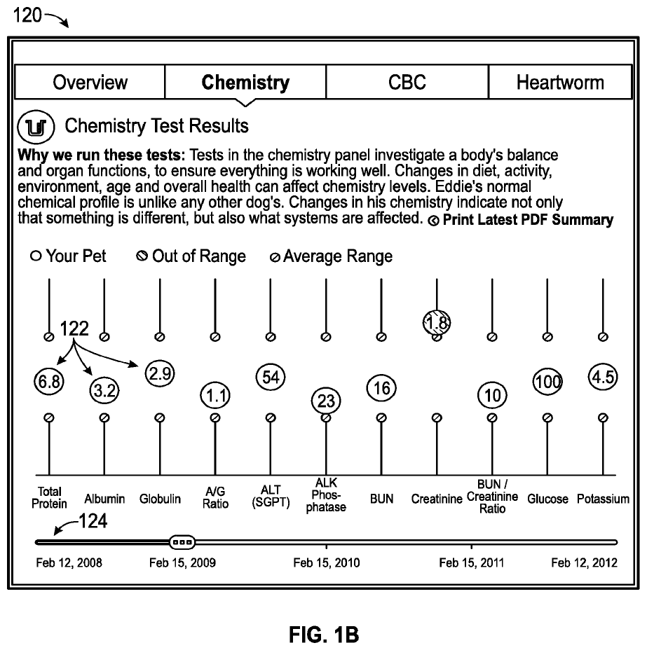

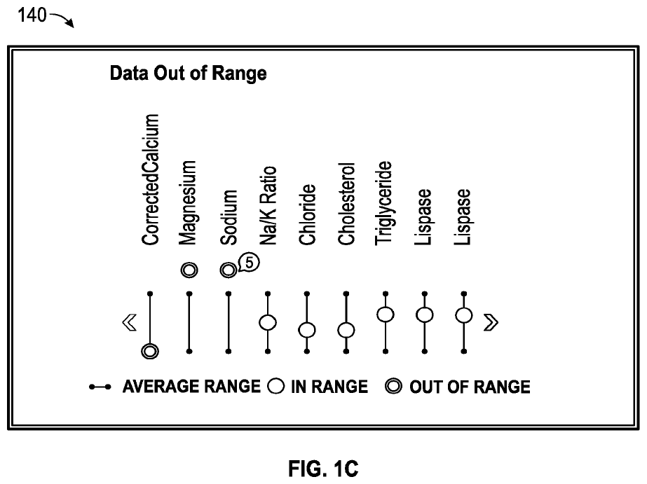

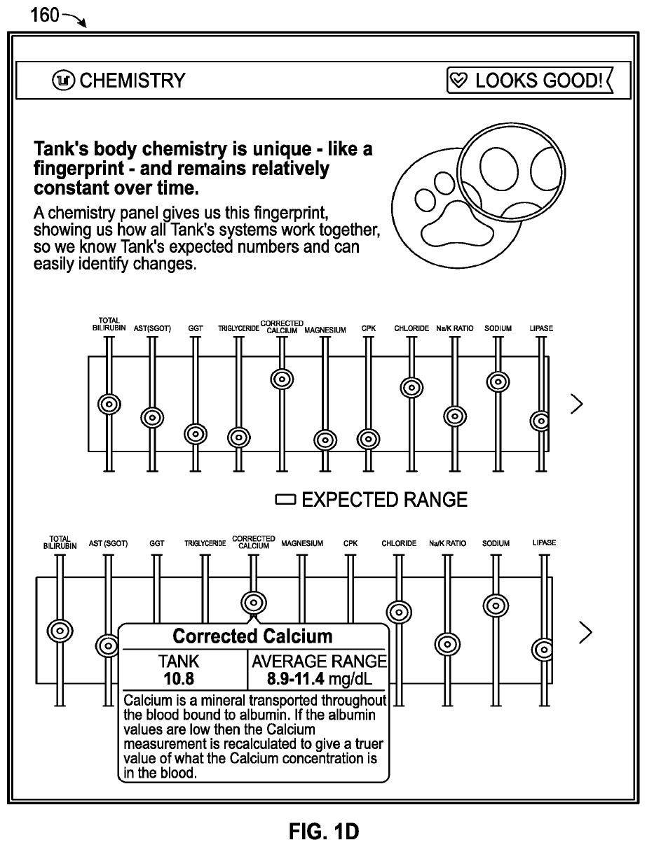

FIG. 1A illustrates an exemplary interface 100 including an informational display of a patient's medical laboratory results, in accordance with one embodiment. In the interface of FIG. 1A, the patient has undergone a blood chemistry test and the results are displayed.

The informational display includes a graphical display (indicated by the number 3) of the patient's test results. The graphical display includes one-dimensional graphs of the quantity of each of the substances tested for. The various substances are listed (indicated by the number 2) above each corresponding one-dimensional graph. As will be readily understood by one of skill in the art, numerous other substances could be included in the informational display, either in addition to or instead of one or more of the substances displayed in FIG. 1A, without deviating from the scope of this disclosure.

The informational display eliminates the need for specialist medical training to appreciate trends in the results and diagnose problematic health conditions. The informational display allows an end-user (patient, caregiver, etc.) to disentangle the statistical lab results and play a role in the assessing and/or the monitoring of a patient's well-being.

By representing the results of the test in this manner, the informational display gives the patient or primary caregiver the ability to appreciate the results of the test in the context of the patient's unique physiology. The patient or primary caregiver need have no formal medical training to participate in the evaluation and tracking of the test results. In the embodiment of FIG. 1A, the quantity of the substance is provided as a one-dimensional graph (3), but other informational diagrams are contemplated. The informational diagrams require no formal medical training to understand, but additional contextual information may be provided to assist an end-user in understanding the health of the patient.

In some embodiments, the informational display provides no numerical values relating to one or more of a patient's test results. Instead, the informational display provides a visualization of the one or more test results by providing a position within a field of expected results. This position may form a pattern over time, and providing an accessible depiction of that pattern may allow the patient or primary caregiver to visually appreciate the patterns in the test results.

In one embodiment, an informational display provides the end-user with the ability to monitor each quantity over time. By providing a layperson-accessible depiction of lab results, the end-user can easily grasp deviations from normal trends when the quantity is displayed over time. FIG. 1B illustrates a chemistry test result display 120 similar to the test results display of FIG. 1A. FIG. 1B also illustrates a timeline 124 below the plurality of informational displays 122 of the quantity of each substance. To visualize trending (and deviations) of test results over time, the end-user can simply move the time line indicator to watch the quantity change from test result to test result. Other variations may use different mechanisms to enable the end-user to visualize the change over time. For example, an animation of the quantity (or quantities) over time could be activated by the end-user.

In some embodiments, the visualization of the test results over time include an animation of the test results. In further embodiments, the animation includes showing a test result from a first period and dissolving that result into a test result from a second period. The end-user may have the ability to control the speed of the “dissolving.” In some embodiments, the end-user may have the ability to continuously loop a test result over a plurality of periods.

By visualizing the test result changes over time, the end-user can monitor for abnormalities in the patient's unique physiology. This may beneficially improve the systems of the prior art, which relied upon comparisons to the greater population. More specifically, although a patient's test results may be within an accepted range vis-à-vis a statistical range or average based on the greater population, deviations within this accepted range may be indicative of early symptoms of a particular illness.

In other embodiments, the informational display may be a two-dimensional representation of the quantity over time. That is, a quantity may be graphed against time to allow the patient to quickly grasp the change in quantity over time and thereby recognize any deviations from normal physiology.

Returning to FIG. 1A, also included in the interface 100 is a heading (indicated by the number 1 at the top of the page). This heading may alert the end-user to the type of test that was performed. To further aid the end-user's understanding, a short description of the test may be included.

Also shown in the informational displays of FIG. 1A is a normal range for each quantity. This normal range may be determined by the patient's historical test results for that same quantity. Other factors may be included in determining the normal range, such as an expected percentage deviation from an average of historical test results. The normal range may also include other patient-specific factors (such as the patient's genetics or demographic) and/or non-patient factors (such as seasonal influences). This normal range may provide another quickly understandable visualization for an end-user.

The normal range can be contrasted with an abnormal range. In this way, the end-user may quickly grasp the patient's test result evolution. A “normal” versus “abnormal” condition may also be referred to as “healthy” versus “sick” or “good” versus “bad” condition. Such characterizations may be viewed by the end-users as accessible and non-judgmental.

The normal range may be characterized as an expected range in some embodiments. The expected range may be determined by looking at other, similarly-matched, patients. This group of patient's may not be delineated into “healthy” and “sick” patients, from which a criteria for healthy is arrived at. Rather, non-parametric statistical distribution tools may be used. For example, the particular test results for the group of patients may be separated into three groups—an expected group (using a predetermined percentage of the population, as arranged by the particular test results), an above-expected group, and a below expected group. In some embodiments, the predetermined percentage is 95%, and the above expected group is the upper 2.5% of the group and the below expected group is the lower 2.5% of the group. For a given patient that is the subject of the informational displays, a test results may be compared to the expected range. The system may not decide whether a result is “healthy” or “sick,” but instead offer the expected range, allowing the end-user to make a decision on the patient's well-being.

The groups described in the foregoing paragraph may be formed by a given patient's individual characteristics. For example, the patient's ethnicity, domicile, sex, age, etc., may be included. In animals, a patient's species or breed may be considered.

To ease the end-user's understanding of the informational displays, a key (indicated by number 4) is provided below the plurality of informational displays. As the key indicates, an out of range of quantity may be highlighted with an enlarged bubble. Such an out of range quantity is illustrated in FIG. 1C as an enlarged bubble (indicated by 5) that is outside the normal range in interface 140.

The interface 100 of FIG. 1A may also allow an end-user to obtain more information regarding a particular test or substance. For example, a roll-over function may enable the end-user to activate an additional informational display that may include background information on a specific test result, such as why the test result is important, what a deviation from normal physiology might mean, what other symptoms may appear for a related illness, and what secondary steps should be taken, for example. In this way, an end-user can determine which aspects of the laboratory results require deeper study. When offered in conjunction with the informational displays described herein, an end-user (patient, patient's primary caregiver, etc.) can educate himself on the particular substance that is out of range and determine whether the deviation from an expected range warrants immediate attention from a doctor or other healthcare professional.

FIG. 1D provides an example of a roll-over function 160, in accordance with one embodiment. The one-dimensional graphs in the upper region provide a depiction similar to that of FIG. 1A. The one-dimensional graphs in the lower region show additional information activated for “corrected calcium.” The activation may have been instigated by an end-user hovering a cursor over the associated graph.

As can be seen in FIG. 1A and FIG. 1C, measured substances in a blood-chemistry test may include corrected calcium, magnesium, sodium, Na/K ratio, chloride, cholesterol, triglyceride, lispase, total protein, albumin, globulin, A/G ratio, ALT, Alk phosphate, BUN, creatinine, BUN/creatinine ratio, glucose, and potassium. As will be readily understood by one of ordinary skill in the art, other substances could be included in a blood-chemistry test without deviating from the scope of this disclosure.

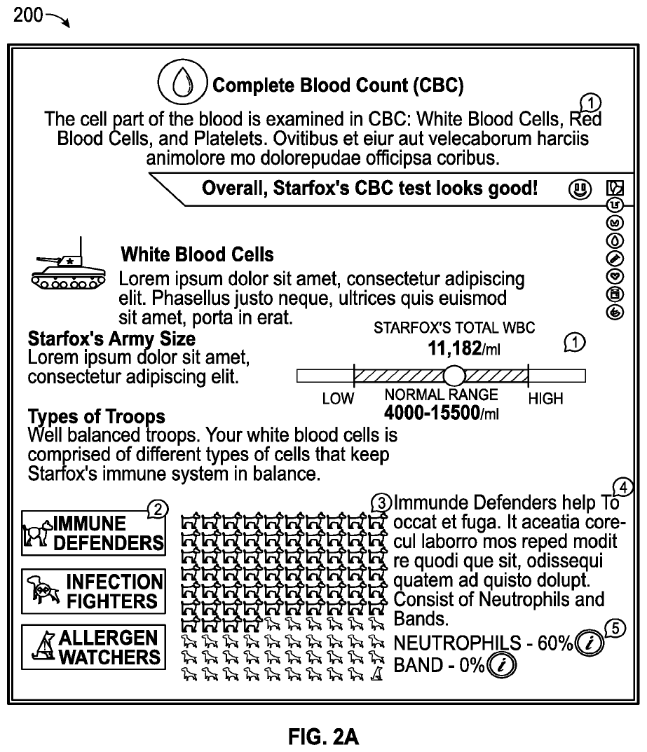

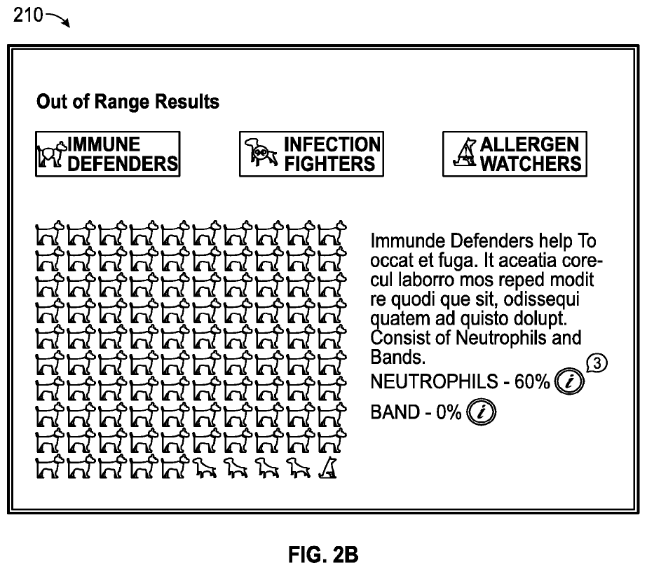

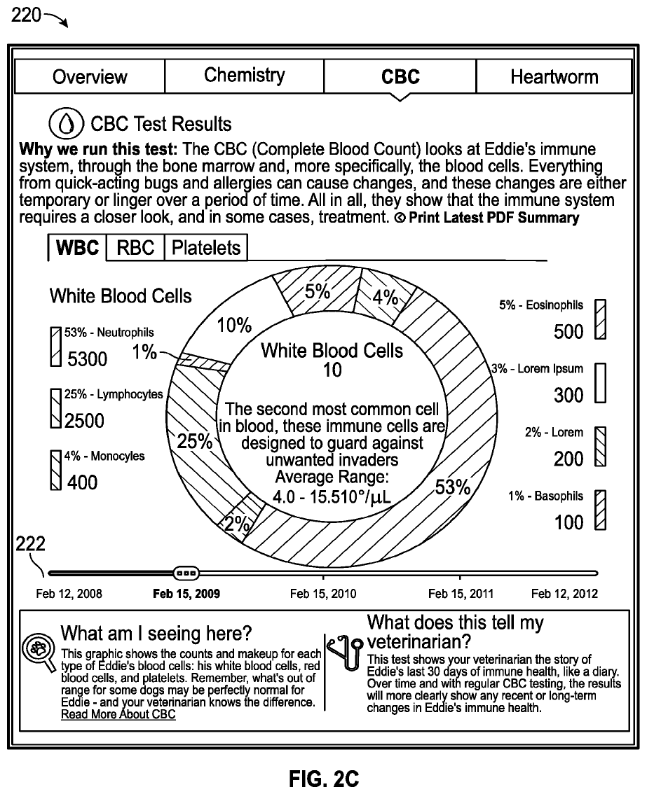

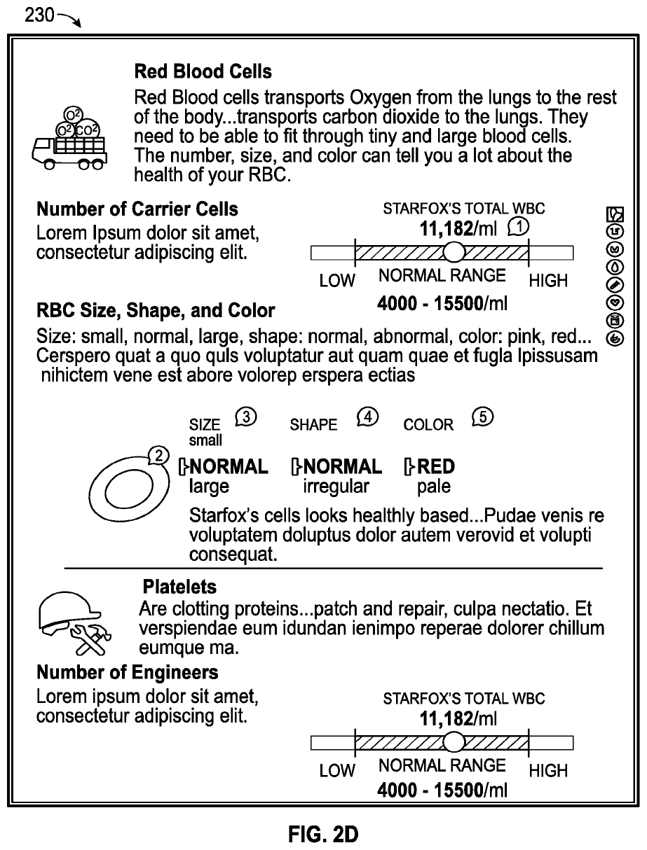

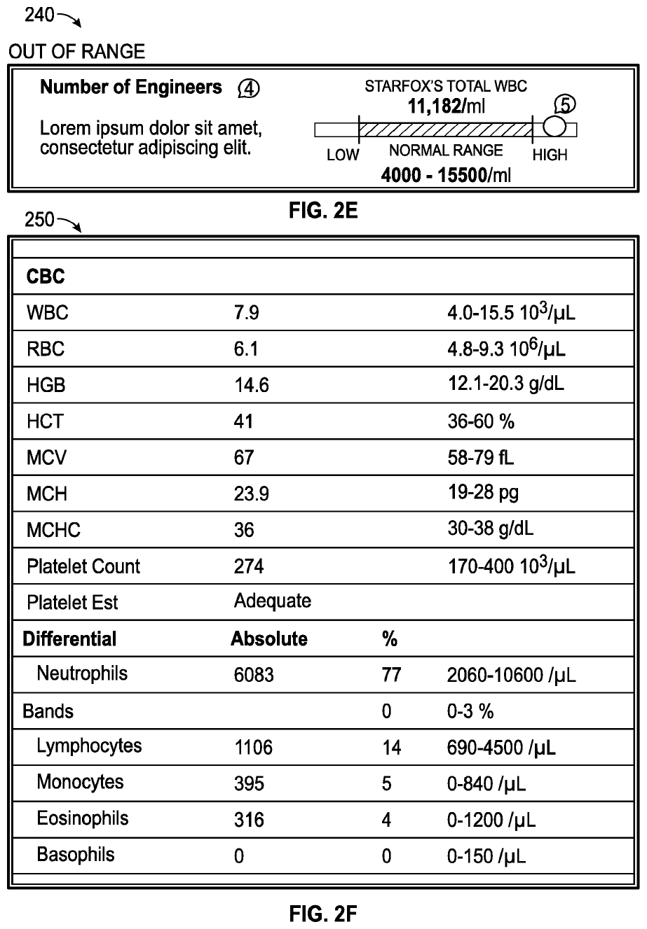

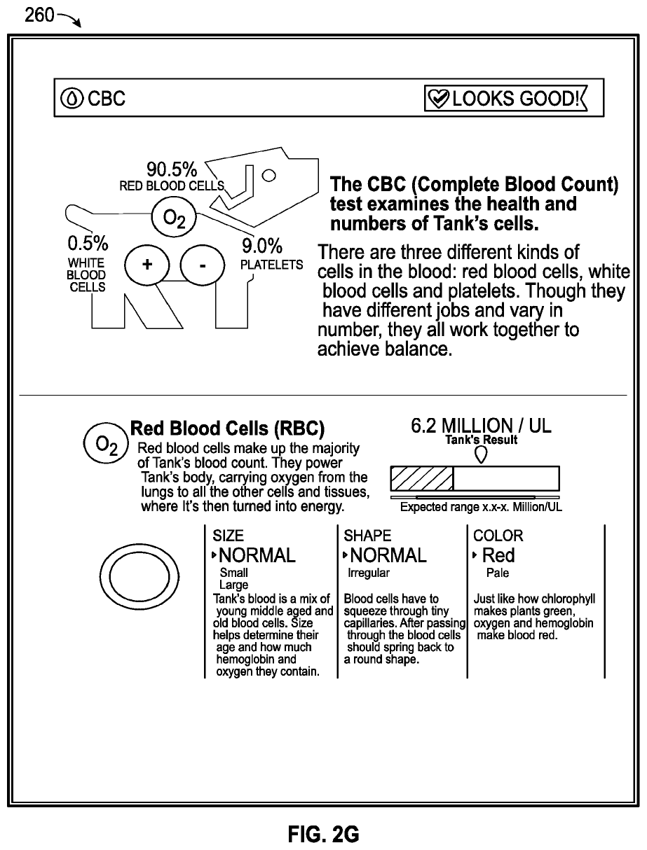

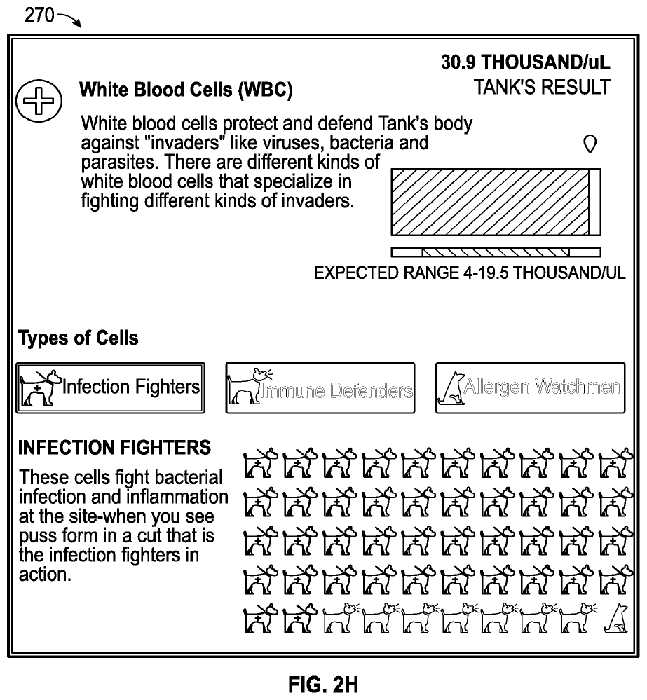

FIG. 2A illustrates another exemplary interface 200 including an informational display of a patient's medical laboratory results, in accordance with one embodiment. In the interface 200 of FIG. 2A, the patient has undergone a complete blood count test (white blood cells, red blood cells, and platelets) and FIG. 2A provides the white blood cell test results (indicated by number 1).

In FIG. 2A, the informational display is arranged as a 10×10 grid (indicated by number 3), with each position on the grid representing one-percent of the total blood-cell count. Each percentage point is represented by a depiction of a corresponding white blood cell-type (immune defender, infection fighter, or allergen watcher) (indicated by number 2). This one-dimensional (scalar) representation of the results of the complete blood count allows the end-users to quickly grasp the significance of the test results.

As with the informational display of FIG. 1A, the informational display of FIG. 2A can include an expected range. An exemplary illustration of a deviation from the expected range is shown in FIG. 2B. In FIG. 2B, an escalated percentage of immune defenders has been detected. The visualization of FIG. 2B enables an end-user to readily appreciate the deviation from normal health parameters.

In a similar manner as the informational display of FIG. 1A, a time evolution 222 of white blood cell test results can be offered to the end-user. FIG. 2C illustrates an interface 220 similar to that of FIG. 2A. Instead of the 10×10 grid, another one-dimensional representation is provided—a donut, where a percentage or the perimeter of the donut corresponds to a percentage of a particular white blood cell type in the patient's blood stream. An indicator on a timeline 222 (illustrated below the donut) allows an end-user to watch the percentage of the white blood cell types change between temporally distinct tests. Other variations may use different mechanisms to enable the end-user to visualize the change over time. For example, an animation of the quantity (or quantities) over time could be activated by the end-user.

In some embodiments, the complete blood count test may include additional results. FIG. 2D illustrates an additional exemplary interface 230 for a complete blood count test, with information provided on red blood cells and platelets, in accordance with one embodiment. An informational display (indicated by number 1) allows a layperson to grasp information about a patient's well-being without formal medical training. FIG. 2E depicts the informational display 240 of FIG. 2D with test results outside a normal range.

FIG. 2F depicts how a doctor may traditionally view a complete blood count test for a patient, in accordance with one embodiment. As can be seen from FIG. 2F, the results require formal medical training to decipher. FIGS. 2G and 2H illustrate exemplary interfaces 260 and 270 for providing complete blood count test to an end-user without formal medical training, in accordance with one embodiment.

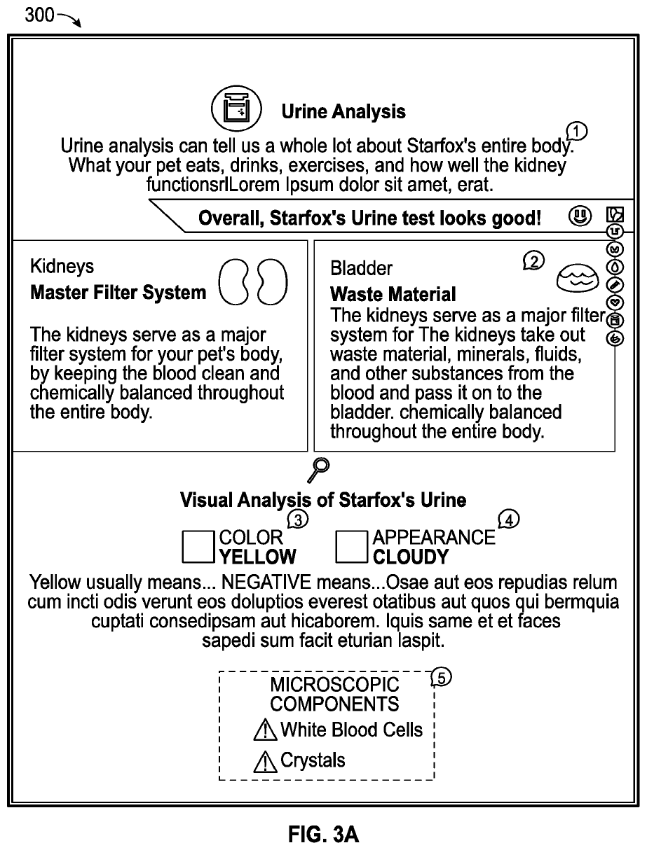

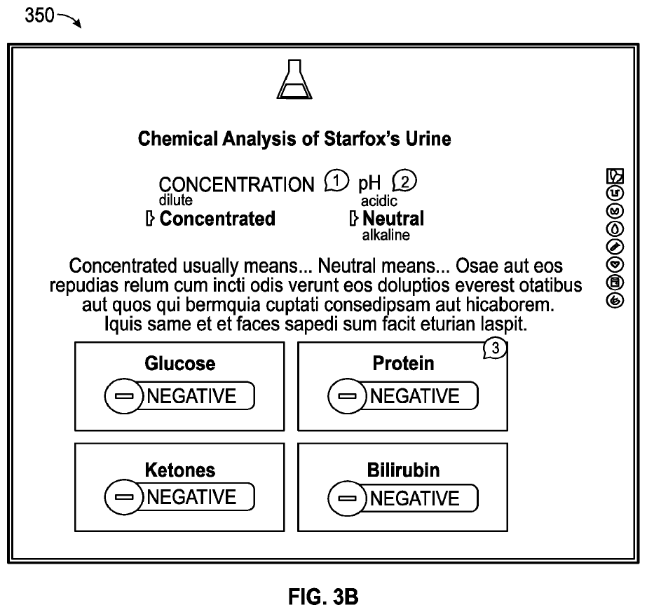

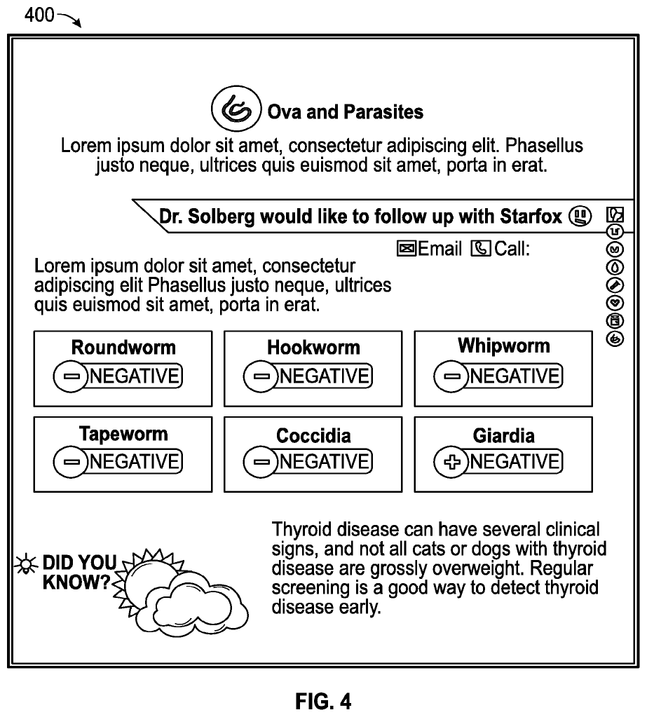

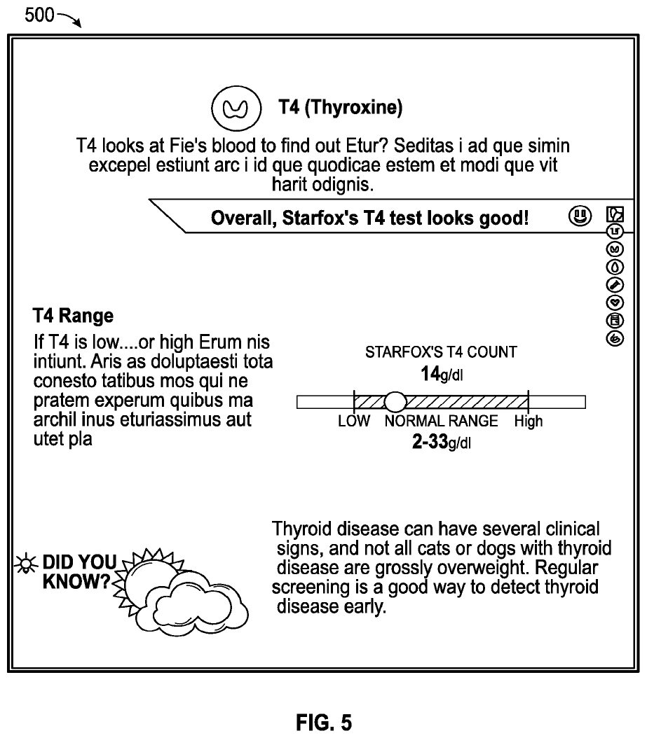

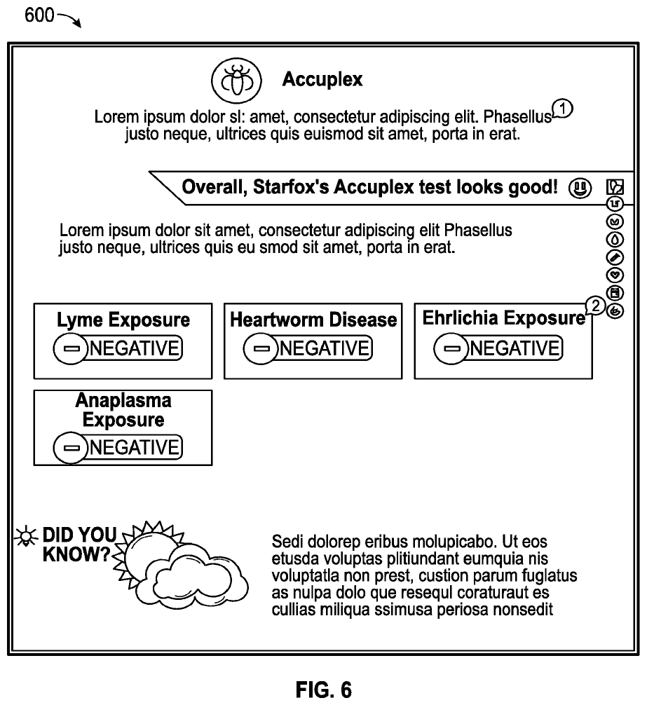

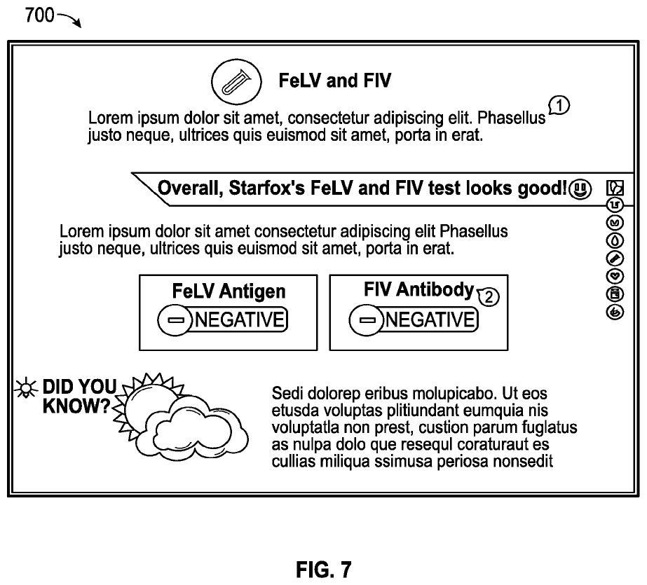

FIGS. 3A-B provide additional exemplary interfaces 300 and 350 that incorporate one or more of the features described above with respect to FIGS. 1A-2E. FIGS. 3A and 3B depict exemplary interfaces 300 and 350 for informational displays of test results for urine analysis, in accordance with one embodiment. FIG. 4 depicts an exemplary interface 400 for informational displays of test results for ova and parasites, in accordance with one embodiment. FIG. 5 depicts an exemplary interface 50 for informational displays of test results for T4 (thyronxine), in accordance with one embodiment. FIG. 6 depicts an exemplary interface 600 for informational displays of test results for accuplex, in accordance with one embodiment. FIG. 7 depicts an exemplary interface 700 for informational displays of test results for FeLV and FIV, in accordance with one embodiment. FIG. 8 depicts an exemplary interface 800 for informational displays of test results for heartworm, in accordance with one embodiment.

Although the figures associated with the above-described exemplary embodiments primarily referred to animals, the disclosure is not so limited As discussed above, the methods and systems described herein may be beneficial to patients (including human patients) their primary caregivers, and others.

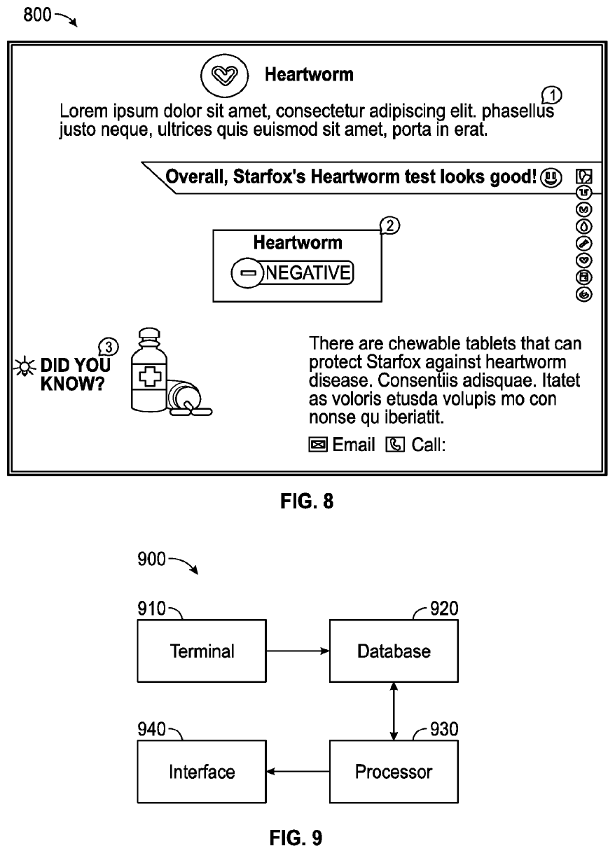

In some variations, the interfaces described above may be implemented in a system for providing medical information to a user, which may include a healthcare provider terminal 910, a patient record database 920, a patient-diagnostic processor 930, and the interface 940, as depicted in FIG. 9. A plurality of laboratory test results may be uploaded via the terminal 910 and may be stored in the patient record database 920. The processor 930 may then use these test results to generate a plurality of informational displays, which may be displayed via the interface 940. In some variations, a computer-implemented method may provide a display of a plurality of informational displays on an interface. In some variations, the interfaces may be displayed using a non-transitory computer storable medium having stored therein instructions, which when executed by a device, cause the device to perform a method of providing medical information to a user. In other variations, an electronic device may have a processor and a memory coupled to the processor to store instructions, which when executed by the processor, cause the processor to perform operations to generate an application programming interface (API) that allows an API-calling component to display a plurality of informational displays on an user interface.

The embodiments described above may operate on one or more server computers that allow interconnected computer network users to participate in a system for providing medical information to a user. This can be accomplished, for example, by program participants accessing non-transitory computer readable media on a server computer via the internet. This readable media contains the program instructions for accomplishing various steps described above. In the context of this document, a computer-readable storage medium can be any medium that can contain or store programming for use by or in connection with an instruction execution system, apparatus, or device. Such computer readable media may be stored on a memory, where a memory is any device capable of storing a computer readable medium and capable of being accessed by a computer. A memory may include additional features. A computer may include a processor. A processor can be any device suitable to access a memory and execute a program stored thereon.

Although the present invention has been fully described in connection with embodiments thereof with reference to the accompanying drawings, it is to be noted that various changes and modifications will become apparent to those skilled in the art. Such changes and modifications are to be understood as being included within the scope of the present invention. The various embodiments of the invention should be understood that they have been presented by way of example only, and not by way of limitation. Although the invention is described above in terms of various exemplary embodiments and implementations, it should be understood that the various features and functionality described in one or more of the individual embodiments are not limited in their applicability to the particular embodiment with which they are described. They instead can be applied, alone or in some combination, to one or more of the other embodiments of the invention, whether or not such embodiments are described, and whether or not such features are presented as being a part of a described embodiment. Thus the breadth and scope of the invention should not be limited by any of the above-described exemplary embodiments.

It will be appreciated that, for clarity purposes, the above description has described embodiments of the invention with reference to different functional units and processors. However, it will be apparent that any suitable distribution of functionality between different functional units, processing logic elements or domains may be used without detracting from the invention. For example, functionality illustrated to be performed by separate processing logic elements, or controllers, may be performed by the same processing logic element, or controller. Hence, references to specific functional units are only to be seen as references to suitable means for providing the described functionality, rather than indicative of a strict logical or physical structure or organization.

As will be readily understood by one of ordinary skill in the art, the interfaces described herein could be utilized on a variety of devices, such as personal computers, laptops, tablets, and smartphones, for example.

As used herein, “a” or “an” means “at least one” or “one or more.” It is understood that aspects and embodiments of the invention described herein include “consisting” and/or “consisting essentially of” aspects and embodiments.

Other objects, advantages and features of the present invention will become apparent from the following specification taken in conjunction with the accompanying drawings.

1. A system for providing medical information to a user comprising:
a terminal to enable uploading of a plurality of laboratory test results, wherein each laboratory test result measures a quantity of each of a respective plurality of substances in a patient;
a database comprising historical laboratory test results associated with the patient;
a processor that generates a plurality of informational displays based on the patient's plurality of laboratory test results and the patient's historical laboratory test results, wherein each informational display provides a visualization of a change in the quantity of the respective substance over a period of time; and
an interface that displays the plurality of displays.

2. The system of claim 1, wherein the visualization of the change in the quantity of the respective substance comprises a plurality of one-dimensional graphs of the quantity, wherein each of the plurality of one-dimensional graphs represents a temporally distinct laboratory test result of the respective substance.

3. The system of claim 1, wherein each of the plurality of informational displays comprises an expected range of the respective substance.

4. The system of claim 3, wherein the expected range is a function of a genetic trait of the patient.

5. The system of claim 1, wherein the plurality of laboratory tests comprises a blood test of the patient and the plurality of substances comprises at least one of total protein, albumin, globulin, A/G ratio, ALT, Alk phosphate, BUN, creatinine, BUN/creatinine ratio, glucose, and potassium.

6. The system of claim 1, wherein the plurality of laboratory tests comprises a blood test of the patient and the plurality of substances comprises at least one of white blood cells, red blood cells, and platelets, a urine analysis of the patient and/or a heartworm test of the patient.

7-8. (canceled)

9. The system of claim 1, further comprising an background information display that provides background information about one of the plurality of test results, wherein the background information display is activated through the interface.

10-18. (canceled)

19. A non-transitory computer readable storage medium having stored therein instructions, which when executed by a device, cause the device to perform a method of providing medical information to a user, the method comprising:
receiving a plurality of test results which measure a quantity of each of a respective plurality of substances in the patient;
accessing historical laboratory test results associated with the patient;
generating a plurality of informational displays based on the patient's plurality of laboratory test results and the patient's historical laboratory test results, wherein each informational display provides a visualization of a change in the quantity of the respective substance over a period of time; and
displaying the plurality of informational displays on an interface.

20. The non-transitory computer readable storage medium of claim 19, wherein the visualization of the change in the quantity of the respective substance comprises a plurality of one-dimensional graphs of the quantity, wherein each the plurality of one-dimensional graphs represents a temporally distinct laboratory test result of the respective substance.

21. The non-transitory computer readable storage medium of claim 19, wherein the method further comprises displaying an expected range of the respective substance on the interface.

22. The non-transitory computer readable storage medium of claim 21, wherein the expected range is a function of a genetic trait of the patient.

23. The non-transitory computer readable storage medium of claim 19, wherein the plurality of laboratory tests comprises a blood test of the patient and the plurality of substances comprises at least one of total protein, albumin, globulin, A/G ratio, ALT, Alk phosphate, BUN, creatinine, BUN/creatinine ratio, glucose, and potassium.

24. The non-transitory computer readable storage medium of claim 19, wherein the plurality of laboratory tests comprises a blood test of the patient and the plurality of substances comprises at least one of white blood cells, red blood cells, and platelets, a urine analysis of the patient and/or a heartworm test of the patient.

25-26. (canceled)

27. The non-transitory computer readable storage medium of claim 19, wherein the method further comprises displaying background information about one of the plurality of test results when the user interacts with the interface.

28. An electronic device, comprising:
a processor to execute instructions; and
a memory coupled with the processor to store instructions, which when executed by the processor, cause the processor to perform operations to generate an application programming interface (API) that allows an API-calling component to perform the following operations:
receiving a plurality of test results which measure a quantity of each of a respective plurality of substances in the patient;
accessing historical laboratory test results associated with the patient;
generating a plurality of informational displays based on the patient's plurality of laboratory test results and the patient's historical laboratory test results, wherein each informational display provides a visualization of a change in the quantity of the respective substance over a period of time; and
displaying the plurality of informational displays on an user interface.

29. The device of claim 28, wherein the visualization of the change in the quantity of the respective substance comprises a plurality of one-dimensional graphs of the quantity, wherein each of the plurality of one-dimensional graphs represents a temporally distinct laboratory test result of the respective substance.

30. The device of claim 28, wherein the operations further comprise displaying an expected range of the respective substance on the user interface.

31. (canceled)

32. The device of claim 28, wherein the plurality of laboratory tests comprises a blood test of the patient and the plurality of substances comprises at least one of total protein, albumin, globulin, A/G ratio, ALT, Alk phosphate, BUN, creatinine, BUN/creatinine ratio, glucose, and potassium.

33. The device of claim 28, wherein the plurality of laboratory tests comprises a blood test of the patient and the plurality of substances comprises at least one of white blood cells, red blood cells, and platelets, a urine analysis of the patient and/or a heartworm test of the patient.

34-35. (canceled)

36. The device of claim 28, wherein the operations further comprise displaying background information about one of the plurality of test results when the user interacts with the interface.
